(19) United States
(12) Patent Application Publication
(10) Pub. No.: US 2018/0271805 A1
(43) Pub. Date: Sep. 27, 2018

(54) COMPOSITION FOR THE TREATMENT OF HUMAN AND MAMMALIAN ATHEROSCLEROSIS AND PATHOLOGICAL CALCIFICATION IN TISSUE

(71) Applicant: NanoBiotech Pharma, Inc., The Villages, FL (US)

(72) Inventor: Gary S. MEZO, The Villages, FL (US)

(73) Assignee: NanoBiotech Pharma, Inc., The Villages, FL (US)

(21) Appl. No.: 15/904,028

(22) Filed: Feb. 23, 2018

Related U.S. Application Data
(60) Provisional application No. 62/477,247, filed on Mar. 27, 2017.

(51) Int. Cl. A61K 31/122; A61K 47/18; A61K 45/06; A23L 33/15

(52) U.S. Cl. CPC A61K 31/122; A61K 47/18; A23V 2200/30; A23L 33/15; A23V 2002/00; A61K 45/06

(57) ABSTRACT

A compound for treatment of a disorder characterized by at least one of pathological calcification and/or plaque formation including a nutraceutical supplement and a sequestrant, wherein the compound does not include an antibiotic.

### PRIORITY

The present application is claims priority to Provisional U.S. Patent Application Ser. No. 62/477,247, having a filing date of Mar. 27, 2017, the disclosure of which is hereby incorporated by reference in its entirety.

### FIELD OF THE INVENTION

The present disclosure generally relates to a therapeutic composition and method for the treatment of pathological calcification, the attendant inflammation, and the resultant soft plaque deposition.

### BACKGROUND OF THE INVENTION

Pathological calcification is the calcification of calcium phosphate or apatite commonly found in human in tissues, tissue beds, organs and intravascular spaces. Calcifying NanoParticles (CNP)-induced pathological calcification begins with the formation of calcium-phosphate deposition around each CNP. CNPs secrete a lipopolysaccharide (LPS) endotoxin biofilm that allows for the protection of the CNPs as well as allowing them to connect, interact, coalesce, move and form aggregates. CNP biofilm LPS endotoxin is a strong mediator of inflammation whenever and wherever expressed from CNPs. This inflammatory response from the affected human (mammal) is seen by swelling as well as spikes in various inflammatory markers, e.g.: matrix metalloproteinases, heat-shock proteins, cytokines, interleukins, mast cells, fibroblasts, T-Lymphocytes, general inflammatory responses, thrombosis or cellular apoptosis. However, currently there are no effective remedies for the treatment of CNP-based diseases.

### SUMMARY OF THE INVENTION

In an aspect, a formulation for treatment or prevention of a disorder characterized by at least one of pathological calcification and plaque formation is provided. The formulation includes a nutraceutical supplement; and a sequestrant, wherein the compound does not include an antibiotic.

In an aspect, the nutraceutical supplement is vitamin K.

In another aspect, the nutraceutical supplement further comprising at least one of Niacin, Pyridoxine HCL (Vitamin B6), Folate, Vitamin C, Selenium, L-Arginine HCL, L-Ornithine HCL, L-Lysine HCL, Bromelain, Trypsin, Papain, Coenzyme Q10, Grapeseed Extract, Hawthorne Berry, Vitamin A, Vitamin E, Vitamin B1, Vitamin B2, Vitamin B12, Magnesium Citrate, Methyl Sulfonyl Methane, Curcuma Longa, Quercitin, Pycnogenol, Gugulipid, Zinc Citrate, mahonia aquifolium, schisandra chinensis, licorice root, alfalfa seed, wheatgrass, green barley grass, chlorella algae, spirulina, flaxseed, milk thistle, aslanguanda, lipase, protease, peptase, serrapeptase, cellulase, and 1-glutathione.

In a further aspect, the sequestrant further comprises at least one of EGTA, DTPA, HEEDTA, CDTA, BAPTA, and pharmaceutically acceptable salts thereof.

In a further aspect, the sequestrant is EDTA and pharmaceutically acceptable salts thereof.

In yet another aspect, the nutraceutical supplement is administered in an amount of from about 5 mg to about 30 mg per pound of body weight.

In an aspect, the sequestrant is administered in an amount of from about 5 mg to about 20 mg per pound of body weight.

In an aspect, the combination of the nutraceutical supplement and the sequestrant is administered in an amount of from about 10 mg to about 50 mg per pound of body weight of a patient.

In an aspect, the nutraceutical supplement and the sequestrant create a mixture having a form of (i) oral dispersible powder or granule, compressed pill or tablet, hard or soft capsule, suspension, lozenges, aqueous or oily suspensions, emulsions, syrup, elixir or sublingual film solution, or timed-release variant of the above or pH dependent controlled-dissolution variant of the above, (ii) finely divided powder or liquid aerosol for inhalation or insufflation, (iii) a sterile aqueous or oil based solution for parenteral administration dosing, and (iv) a suppository.

In another aspect, the sequestrant is administered separately from the nutraceutical supplement and is in a form of suppository and is administered at about 9.5 mg per pound of body weight.

In a further aspect, the weight ratio of the nutraceutical supplement to the sequestrant is from about 2 to about 1.

In yet another aspect, the formulation further comprising a time-release capsule containing a mixture of the nutraceutical supplement and the sequestrant.

In an aspect, a formulation for treatment of a disorder characterized by at least one of pathological calcification and plaque formation is provided. The formulation includes vitamin K; and a sequestrant comprising at least one of EDTA, EGTA, DTPA, HEEDTA, CDTA, BAPTA, and pharmaceutically acceptable salts thereof, wherein the compound does not include an antibiotic.

In another aspect, wherein the vitamin K is administered in an amount of from about 1 mg to about 3 mg per pound of body weight.

In a further aspect, wherein at least one of EDTA, EGTA, DTPA, HEEDTA, CDTA, and BAPTA is administered in an amount of from about 5 mg to about 20 mg per pound of body weight.

In yet another aspect, the formulation further includes at least one of Niacin, Pyridoxine HCL (Vitamin B6), Folate, Vitamin C, Selenium, L-Arginine HCL, L-Ornithine HCL, L-Lysine HCL, Bromelain, Trypsin, Papain, Coenzyme Q10, Grapeseed Extract, Hawthorne Berry, Vitamin A, Vitamin E, Vitamin B1, Vitamin B2, Vitamin B12, Magnesium Citrate, Methyl Sulfonyl Methane, Curcuma Longa, Quercitin, Pycnogenol, Gugulipid, Zinc Citrate, mahonia aquifolium, schisandra chinensis, licorice root, alfalfa seed, wheatgrass, green barley grass, chlorella algae, spirulina, flaxseed, milk thistle, aslanguanda, lipase, protease, peptase, serrapeptase, cellulase, and 1-glutathione.

In an aspect, the weight ratio of the vitamin K to one or more of EDTA, EGTA, DTPA, HEEDTA, CDTA, and BAPTA is from about 1:30 to about 1:60.

In another aspect, a method for treating and preventing a disorder characterized by at least one of pathological calcification and plaque is provided. The method includes administrating to a patient therapeutic formulation in effective amounts including vitamin K; and a sequestrant, wherein the therapeutic formulation is devoid of antibiotics.

In an aspect, the weight ratio of the vitamin K to sequestrant is from about 1:30 to about 1:60.

Additional features and advantages of various embodiments will be set forth, in part, in the description that follows, and will, in part, be apparent from the description, or may be learned by the practice of various embodiments. The objectives and other advantages of various embodiments will be realized and attained by means of the elements and combinations particularly pointed out in the description herein.

### DETAILED DESCRIPTION OF THE INVENTION

It is to be understood that both the foregoing general description and the following detailed description are exemplary and explanatory only, and are intended to provide an explanation of various embodiments of the present teachings.

In its broad and varied embodiments, the invention provides a therapeutic formulation and method for treatment and/or prevention of a disorder characterized by at least one of pathological calcification and plaque formation. The compound can include a nutraceutical supplement and a sequestrant, such as a metal sequestrant, without having any antibiotics. The studies in the examples illustrate that by eliminating antibiotics, the compound has synergistic effects.

The combination of a nutraceutical supplement and a sequestrant may be useful in the treatment of other Nanobacteria/Calcifying NanoParticles (NB/CNP) related/pathological calcification conditions, including but not limited to, for example, heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

In an aspect, the nutraceutical supplement can be vitamin K. In addition or alternatively, the nutraceutical supplement can be any one or more of niacin, vitamin B6 (Pycnogenol), folate, vitamin C, selenium, L-arginine, L-ornithine, L-lysine, bromelain, trypsin, papain, coenzyme-Q10, grapeseed extract, hawthorne berry, vitamin A, vitamin E, vitamin B1, vitamin B2, vitamin B12, magnesium citrate, methyl sulfonyl methane, curcuma longa, quercitin, pycnogenol, gugulipid, zinc citrate, herbal extracts, such as mahonia aquifolium, schisandra chinensis, licorice root, alfalfa seed, wheatgrass, green barley grass, chlorella algae, spirulina, flaxseed, milk thistle, aslanguanda, and other enzymes, such as lipase, protease, peptase, serrapeptase, cellulase, 1-glutathione.

In an aspect, the sequestrant can be one or more of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof. For example, suitable EDTA salt sequestrates include, but are not limited to, Calcium EDTA, Sodium EDTA, Calcium Disodium EDTA, Dicalcium Disodium EDTA.

In an aspect, the quantity of each component that make up the nutraceutical supplement as well as the quantity of the nutraceutical supplement used may be varied for different patients and/or treatment conditions. Generally, a person may be required to take from about 1 mg to about 70 mg per pound body weight of the nutraceutical supplement. For example, a person may be required to take from about 2 mg to about 60 mg, from about 3 mg to about 50 mg, such as from about 4 mg to about 40 mg, for example, from about 5 mg to about 30 mg per pound of bodyweight of the nutraceutical supplement. In an aspect, one or more of the components in the nutraceutical supplement can make up from about 1% or less to about 99% or more of the nutraceutical supplement. For example, the one or more components can make up from about 5% to about 95%, from about 10% to about 90% from about 20% to about 80% from about 30% to about 70%, from about 40% to about 60%, or about 50% of the nutraceutical supplement.

In an aspect, the quantity of each component that make up the sequestrant as well as the quantity of the sequestrant used may be varied for different patients and/or treatment conditions. Generally, a person may be required to take from about 1 mg to about 60 mg per pound body weight of the sequestrant. For example, a person may be required to take from about 2 mg to about 50 mg, from about 3 mg to about 40 mg, from about 4 mg to about 30 mg, such as from about 5 mg to about 20 mg per pound of bodyweight of the sequestrant. In an aspect, one or more of the components in the sequestrant can make up from about 1% or less to about 99% or more of the sequestrant. For example, the one or more components can make up from about 5% to about 95%, from about 10% to about 90% from about 20% to about 80% from about 30% to about 70%, from about 40% to about 60%, or about 50% of the sequestrant.

In an aspect, the nutraceutical supplement to the sequestrant ratio can be from about 10:1 to about 1:10, such as from about 8:1 to about 1:8, for example from about 7:1 to about 1:7, from about 6:1 to about 1:6, from about 5:1 to about 1:5, from about 4:1 to about 1:4, from about 3;1 to about 1:3, such as from about 2:1 to about 1:2, for example a ratio of about 1:1.

In an aspect, the nutraceutical supplement and the sequestrant create a mixture having a form of oral dispersible powder or granule, compressed pill or tablet, hard or soft capsule, suspension, lozenges, aqueous or oily suspensions, emulsions, syrup, elixir or sublingual film or solution, or timed-release variant of the above or pH dependent controlled-dissolution variant of the above. In another aspect, nutraceutical supplement and the sequestrant create a mixture having a form of finely divided powder or liquid aerosol for inhalation or insufflation. In another aspect, the nutraceutical supplement and the sequestrant create a mixture having a form of a sterile aqueous or oil based solution for parenteral administration dosing. In another aspect, the nutraceutical supplement and the sequestrant create a mixture having a form that allows the formulation to be administered via suppository.

In an aspect, nutraceutical supplement and the sequestrant can be administered separately. For example, one of the sequestrant and the nutraceutical supplement can be in a form of oral dispersible powder or granule, compressed pill or tablet, hard or soft capsule, suspension, lozenges, aqueous or oily suspensions, emulsions, syrup, elixir or sublingual film or solution, or timed-release variant of the above or pH dependent controlled-dissolution variant of the above, while the other one of sequestrant and the nutraceutical supplement can be in a form that allows the formulation to be administered via suppository, parenteral dosing, or inhalation or insufflation. One skilled in the art would understand that any combination dosing method would work so long as both the nutraceutical supplement and the sequestrant are administered to a patient.

In an aspect, a 160 pound subject can orally consume 8 capsules at any time, for example, at bedtime. Each of the 8 capsules were size “00” and contained approximately 746 mg of formulation In an aspect, each of the 8 capsules should be taken within no more than a few minutes apart and with an adequate amount of clear water or apple juice prior to sleeping. In an aspect, a subject weighing over or less than 160 pounds should increase or decrease the dosage by 1 capsule per each 20 pounds. Thus, a person weighing approximately 220 pounds (i.e., 60 pounds over the 160 pounds) should consume three additional capsules for a total of 11 capsules. Similarly, a person weighing approximately 140 pounds (i.e., 20 pounds under the 160 pounds) should consume one fewer capsule for a total of 7 capsule.

In an aspect, the capsules are designed to be absorbed into the small bowel. In an example, to accomplish the absorption of the capsules in the small bowel, the capsules can be designed to have a pH-dependent dissolution of from about 6.0 to about 6.5.

### EXAMPLES

### Example 1

Ninety one patients with stable coronary artery disease (CAD) and positive Coronary Artery Calcification (CAC) scores were enrolled into a 3 month treatment regimen pilot study that included daily administration of two-component composition composed of (1) Nutraceutical Powder (Vitamin C, Vitamin B6, Niacin, Folic Acid, Selenium, EDTA, 1-Arginine, 1-Lysine, 1-Ornithine, Bromelain, Trypsin, CoQ10, Grapeseed Extract, Hawthorn Berry, Papain) 5 cm3 taken orally every evening; and (2) EDTA 1500 mg taken in a rectal suppository base every evening. Exclusion criteria included: (1) zero CAC score, (2) recent (<30 days) major adverse cardiac event, (3) women of childbearing age, (4) recent diagnosis of thyroid or parathyroid disease, (5) clinically significant renal insufficiency or liver function abnormalities, and (6) recent (<30 days) acute congestive heart failure. Other than discontinuing any herbal or vitamin preparation, patients maintained their normal medical regime during the study. Baseline History and Physical examination were performed. The same CAC scoring machine was used for each individual patient to assess initial and final CAC scores.

100% of the patients completed the study. The 91 patients' CAC scores decreased by an average of 58.5%. Patients that had previously experienced anginal chest pain upon daily activities reported that their anginal symptoms had ceased and their tolerance to exercise had improved.

### COMPARATIVE EXAMPLE

### Example 2

A substantially similar study as in Example 1 was conducted with 100 patients with stable coronary artery disease (CAD) and positive Coronary Artery Calcification (CAC) scores were enrolled into a 4 month treatment regimen that included daily administration of the two-component composition described in Example 1 and an additional composition composed of Tetracycline HCl 500 mg taken orally every evening. In addition to the exclusion criteria described in Example 1, patients having known tetracycline allergy were excluded from the study. Before completion of the study, one patient withdrew secondary to a presumed sensitivity to tetracycline HCL and twenty-two patients were withdrawn due to noncompliance. All other criteria remained substantially identical as in Example 1.

Of the remaining 77 patients completing the study, their CAC scores decreased by an average of 14% after 4 months compared to the CAC score in the pilot study decrease of 58.5% after 3 months.

The addition of the tetracycline was the only difference & gross variable that could be attributed to the 44.5% decreased performance. Further study of the tetracycline molecule itself illustrated that it has 6 binding sites on the exterior of the molecule and that it has a very high affinity for binding to calcium, as such it may prevent removal of calcification in the intimal-medial space of the coronary artery arteries.

From the foregoing description, those skilled in the art can appreciate that the present teachings can be implemented in a variety of forms. Therefore, while these teachings have been described in connection with particular embodiments and examples thereof, the true scope of the present teachings should not be so limited. Various changes and modifications may be made without departing from the scope of the teachings herein.

This scope disclosure is to be broadly construed. It is intended that this disclosure disclose equivalents, means, systems and methods to achieve the devices, activities and mechanical actions disclosed herein. For each device, article, method, mean, mechanical element or mechanism disclosed, it is intended that this disclosure also encompass in its disclosure and teaches equivalents, means, systems and methods for practicing the many aspects, mechanisms and devices disclosed herein. Additionally, this disclosure regards a coating and its many aspects, features and elements. Such a device can be dynamic in its use and operation, this disclosure is intended to encompass the equivalents, means, systems and methods of the use of the device and/or article of manufacture and its many aspects consistent with the description and spirit of the operations and functions disclosed herein. The claims of this application are likewise to be broadly construed.

The description of the inventions herein in their many embodiments is merely exemplary in nature and, thus, variations that do not depart from the gist of the invention are intended to be within the scope of the invention. Such variations are not to be regarded as a departure from the spirit and scope of the invention

We claim:

1. A formulation for treatment or prevention of a disorder characterized by at least one of pathological calcification and/or plaque formation comprising:
a nutraceutical supplement; and
a sequestrant,
wherein the formulation does not include an antibiotic.

2. The formulation of claim 1, wherein the nutraceutical supplement is vitamin K.

3. The formulation of claim 2, wherein the nutraceutical supplement further comprising at least one of Niacin, Pyridoxine HCL (Vitamin B6), Folate, Vitamin C, Selenium, L-Arginine HCL, L-Ornithine HCL, L-Lysine HCL, Bromelain, Trypsin, Papain, Coenzyme Q10, Grapeseed Extract, Hawthorne Berry, Vitamin A, Vitamin E, Vitamin B1, Vitamin B2, Vitamin B12, Magnesium Citrate, Methyl Sulfonyl Methane, Curcuma Longa, Quercitin, Pycnogenol, Gugulipid, Zinc Citrate, mahonia aquifolium, schisandra chinensis, licorice root, alfalfa seed, wheatgrass, green barley grass, chlorella algae, spirulina, flaxseed, milk thistle, aslanguanda, lipase, protease, peptase, serrapeptase, cellulase, and 1-glutathione.

4. The formulation of claim 1, wherein the sequestrant further comprises at least one of EGTA, DTPA, HEEDTA, CDTA, BAPTA, and pharmaceutically acceptable salts thereof.

5. The formulation of claim 1, wherein the sequestrant is EDTA and pharmaceutically acceptable salts thereof.

6. The formulation of claim 1, wherein the nutraceutical supplement is administered in an amount of from about 5 mg to about 30 mg per pound of body weight.

7. The formulation of claim 1, wherein the sequestrant is administered in an amount of from about 5 mg to about 20 mg per pound of body weight.

8. The formulation of claim 1, wherein the combination of the nutraceutical supplement and the sequestrant is administered in an amount of from about 10 mg to about 50 mg per pound of body weight of a patient.

9. The formulation of claim 1, wherein the nutraceutical supplement and the sequestrant create a mixture having a form of (i) oral dispersible powder or granule, compressed pill or tablet, hard or soft capsule, suspension, lozenges, aqueous or oily suspensions, emulsions, syrup, elixir or sublingual solution, sublingual film, or timed-release variant of the above or pH dependent controlled-dissolution variant of the above, (ii) finely divided powder or liquid aerosol for inhalation or insufflation, (iii) a sterile aqueous or oil based solution for parenteral administration dosing, and (iv) a suppository.

10. The formulation of claim 1, wherein the sequestrant is administered separately from the nutraceutical supplement and is in a form of suppository and is administered at about 9.5 mg per pound of body weight.

11. The formulation of claim 1, wherein a weight ratio of the nutraceutical supplement to the sequestrant is from about 2 to about 1.

12. The formulation of claim 1, further comprising a time-release capsule containing a mixture of the nutraceutical supplement and the sequestrant.

13. A formulation for treatment of a disorder characterized by at least one of pathological calcification and plaque formation comprising:
a vitamin K; and
a sequestrant comprising at least one of EDTA, EGTA, DTPA, HEEDTA, CDTA, BAPTA, and pharmaceutically acceptable salts thereof,
wherein the formulation does not include an antibiotic.

14. The formulation of claim 13, wherein the vitamin K is administered in an amount of from about 1 mg to about 3 mg per pound of body weight.

15. The formulation of claim 13, wherein at least one of EDTA, EGTA, DTPA, HEEDTA, CDTA, and BAPTA is administered in an amount of from about 5 mg to about 20 mg per pound of body weight.

16. The formulation of claim 13, further comprising at least one of Niacin, Pyridoxine HCL (Vitamin B6), Folate, Vitamin C, Selenium, L-Arginine HCL, L-Ornithine HCL, L-Lysine HCL, Bromelain, Trypsin, Papain, Coenzyme Q10, Grapeseed Extract, Hawthorne Berry, Vitamin A, Vitamin E, Vitamin B1, Vitamin B2, Vitamin B12, Magnesium Citrate, Methyl Sulfonyl Methane, Curcuma Longa, Quercitin, Pycnogenol, Gugulipid, Zinc Citrate, mahonia aquifolium, schisandra chinensis, licorice root, alfalfa seed, wheatgrass, green barley grass, chlorella algae, spirulina, flaxseed, milk thistle, aslanguanda, lipase, protease, peptase, serrapeptase, cellulase, and 1-glutathione.

17. The formulation of claim 13, wherein a weight ratio of the vitamin K to one or more of EDTA, EGTA, DTPA, HEEDTA, CDTA, and BAPTA is from about 1:30 to about 1:60.

18. A method for treating and preventing a disorder characterized by at least one of pathological calcification and plaque, the method comprising:
administrating to a patient the formulation of claim 1 in effective amounts comprising:
a vitamin K; and
a sequestrant,
wherein the formulation is devoid of antibiotics.

19. The method of claim 18, wherein a weight ratio of the vitamin K to sequestrant is from about 1:30 to about 1:60.
